(19) United States
(12) Patent Application Publication
(10) Pub. No.: US 2024/0397902 A1
(43) Pub. Date: Dec. 5, 2024

(54) METHOD AND SYSTEM FOR EARLY DETECTION OF BOVINE MASTITIS USING ENHANCED CHEMILUMINESCENCE

(71) Applicant: The State of Israel, Ministry of Agriculture & Rural Development, Agricultural Research Organization, Rishon Lezion (IL)

(72) Inventors: Giorgi SHTENBERG, Herzliya (IL); Narsingh R. NIRALA, Bihar (IN)

(21) Appl. No.: 18/700,312

(22) PCT Filed: Oct. 11, 2022
(86) PCT No.: PCT/IL2022/051078

§ 371 Date: Apr. 11, 2024

Related U.S. Application Data
(60) Provisional application No. 63/254,189, filed on Oct. 11, 2021.

(51) Int. Cl. A01J 5/013; G01N 33/553; G01N 33/72

(52) U.S. Cl. CPC A01J 5/013; G01N 33/553; G01N 33/725; G01N 2800/365

(57) ABSTRACT

A method and system for early detection of Bovine Mastitis (BM) uses enhanced chemiluminescence (CL) to determine Haptoglobin (Hp) levels in highly diluted milk samples. The samples are placed in the wells of one or more bio-functionalized Hemoglobin (Hb) modified CL assay plates. Hp-Hb binding occurs in those samples containing Hp. After a first pre-determined time duration, the wells are treated with a CL solution containing luminol and peroxide, and a colloidal suspension of crosslinked nanoparticles. After a second pre-determined time duration, the CL intensity approaches a steady state value. A processor analyzes CL images provided by a camera in order to measure CL intensity and to determine an estimate of Hp level in each well, based on a pre-determined regression curve. The processor then forms a BM clinical diagnosis by combining the estimated HP levels from wells with different milk sample dilutions.

### CROSS-REFERENCE TO RELATED APPLICATIONS

This application is related to and claims priority from commonly owned U.S. Provisional Patent Application No. 63/254,189, entitled “System and Method for Early Detection of Bovine Mastitis using Enhanced Chemiluminescence”, filed on Oct. 11, 2021, the disclosure of which is incorporated by reference in its entirety herein.

### TECHNICAL FIELD

The present invention relates to methods and systems for early detection of Bovine Mastitis (BM) using chemiluminescence.

### BACKGROUND OF THE INVENTION

BM is a common disease in dairy animals, leading to a decrease in milk production and to increased veterinary costs in maintaining the health of a herd. Typically, BM is diagnosed by estimating the somatic cell count (SCC) in plasma or milk samples, based upon an assay which measures the concentration of one or more biomarkers. One such biomarker, Haptoglobin (Hp) acute phase protein, has been found to increase by a factor of ten in quarters affected by BM, as compared with healthy quarters. Milk Hp is traditionally detected by commercial immunoassays based on hemoglobin (Hb) binding capacity, e.g. Enzyme-Linked Immunosorbent Assay (ELISA); however such methods are often too cumbersome, expensive, and/or time-consuming for on-site herd maintenance.

U.S. Pat. No. 10,866,250, due to Lehmann et al., dated Dec. 15, 2020, and entitled “Method and Apparatus for Monitoring the State of Health of Dairy Cows”, discloses methods and apparatuses for monitoring the state of health of dairy cows based on analyzing the Hp biomarker and part of the polymeric immunoglobulin receptor (PIGR), the secretory component (SC), in a milk sample. This allows diagnosis of mastitis or systemic diseases which occur outside the udder on the basis of the protein biomarker described here.

Promising research results for BM detection based upon a chemiluminescence (CL) assay have appeared in an article by N. R. Nirala et al., entitled “Milk Haptoglobin Detecting Based on Enhanced Chemiluminescence of Gold Nanoparticles”, published in Talanta vol. 197, pp. 257-263, 2019. In this technique, the presence of Hp protein in a milk sample is indicated by a decrease in the CL emission of light at a specific wavelength, when the sample is mixed with a liquid-phase biofunctionalized assay containing a luminol-oxidant-Hb system and gold nanoparticles.

### SUMMARY OF THE INVENTION

The present invention is directed to a label-free system and method for early detection of BM using enhanced CL. Due to its extreme sensitivity to Hp, even in highly-diluted milk samples, the method provides reliable BM diagnosis of sub-clinical as well as clinical cases of BM in dairy animals.

According to one aspect of the presently disclosed subject matter, there is provided a method for early detection of Bovine Mastitis using enhanced chemiluminescence. The method includes the steps of: (a) preparing one or more bio-functionalized Hemoglobin (Hb)-modified assay plates, a chemiluminescent (CL) solution, a peroxide solution and a camera; (b) collecting milk samples and preparing a multiplicity of sample dilutions; (c) applying sample dilutions to wells of the assay plate(s) and waiting a first pre-determined time interval; (d) adding CL and peroxide solutions to the wells and waiting a second pre-determined time interval; (e) acquiring one or more CL images of the assay plate(s); (f) analyzing each CL image to determine a CL intensity measurement for each well; and (g) estimating a Haptoglobin (Hp) level for each well of the assay plate(s) and combining the estimated Hp levels to form a BM clinical diagnosis.

According to some aspects, the CL solution includes luminol and a colloidal suspension of nanoparticles.

According to some aspects, the nanoparticles include one or more materials selected from a group consisting of gold, magnetite (Fe₃O₄) and zinc oxide (ZnO).

According to some aspects, the CL solution includes a 1,3-propanedithiol (PDT) solution in methanol.

According to some aspects, the camera is sensitive to CL blue light emitted in a wavelength range that includes 425 nanometers.

According to some aspects, the camera is fitted with a selective blue filter and/or a shroud.

According to some aspects, the sample dilutions differ in dilution ratio by at least an order of magnitude.

According to some aspects, at least one of the wells of the assay plate(s) is a control well, corresponding to no significant BM disease.

According to some aspects, an Hp-Hb binding reaction occurs during the first pre-determined time interval.

According to some aspects, the first pre-determined time interval is less than or equal to 30 minutes.

According to some aspects, a CL emission intensity approaches a steady-state during the second pre-determined time interval.

According to some aspects, the second pre-determined time interval is less than or equal to ten minutes.

According to some aspects, the CL intensity measurement is determined by averaging over a region of interest in the CL image.

According to some aspects, the estimating of the Hp level utilizes a pre-determined regression curve.

According to some aspects, the BM clinical diagnosis corresponds to a BM disease level selected from a group consisting of no significant disease, sub-clinical disease, and clinical disease.

According to another aspect of the presently disclosed subject matter, there is provided a system for early detection of Bovine Mastitis using enhanced chemiluminescence. The system includes: one or more bio-functionalized Hemoglobin (Hb)-modified assay plate(s), each plate having a multiplicity of wells containing different milk sample dilutions; a chemiluminescence (CL) solution and a peroxide solution for producing an emission of CL light by the milk sample dilution in each of the wells; a camera configured to receive the emission of CL light and to produce CL images; and a processor. The processor is configured to analyze the CL images to determine a CL intensity measurement and an estimate of Haptoglobin (Hp) level, for each well of the assay plate(s).

According to some aspects, the CL solution comprises luminol and a colloidal suspension of nanoparticles.

According to some aspects, at least one of the wells of the assay plate(s) is a control well, corresponding to no significant BM disease.

According to some aspects, the processor determines a CL intensity measurement for each well by averaging over a region of interest of the CL image.

According to some aspects, the processor utilizes a pre-determined regression curve to determine the estimates of Hp level.

According to some aspects, the processor is configured to combine the estimates of Hp level to form a BM clinical diagnosis.

According to some aspects, the BM clinical diagnosis corresponds to a BM disease level selected from a group consisting of no significant disease, sub-clinical disease, and clinical disease.

According to some aspects, the processor and the camera are integral components of a smartphone.

According to some aspects, the camera includes a charge-coupled device (CCD) sensor, a complementary metal-oxide-semiconductor (CMOS) sensor, or a photomultiplier.

### BRIEF DESCRIPTION OF THE DRAWINGS

Some embodiments of the present invention are herein described, by way of example only, with reference to the accompanying drawings. With regard to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1: An exemplary schematic of a system for early detection of BM, according to the invention.

FIG. 2A: An exemplary CL image of an assay plate with various dilutions and Hp concentrations.

FIG. 2B: An exemplary graph of CL intensity versus dilution and Hp concentration obtained by analysis of the CL image of FIG. 2A.

FIG. 3: An exemplary block diagram of a method for early detection of BM, according to the invention.

### DETAILED DESCRIPTION OF THE INVENTION

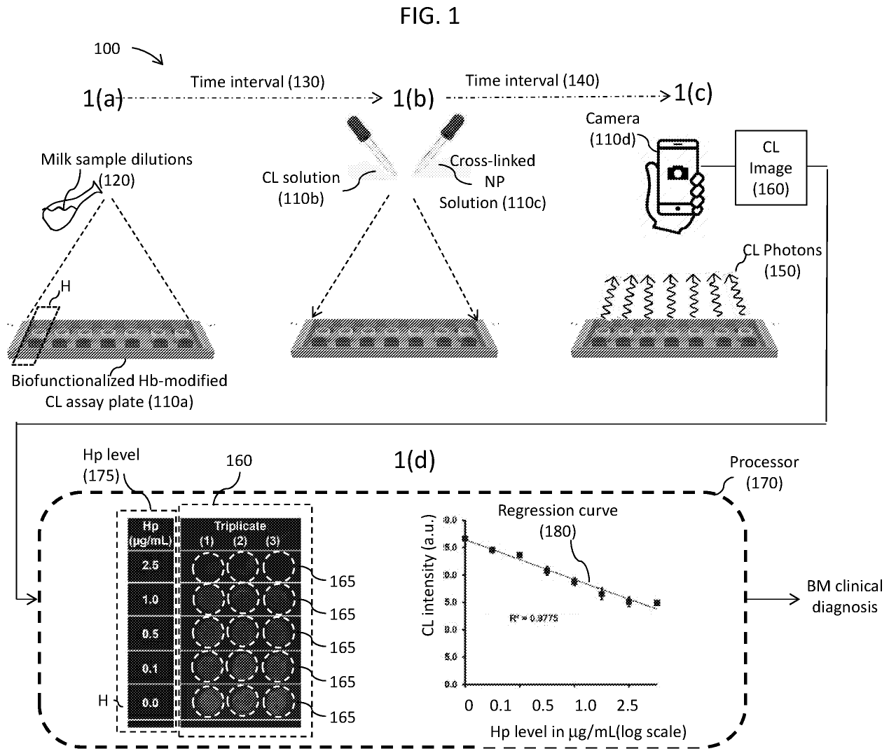

FIG. 1 shows an exemplary schematic of a system 100 for early detection of BM, according to the invention. A CL kit 110 is prepared in advance containing:
110a: one or more bio-functionalized Hb-modified CL assay plates;
110b: a CL solution containing luminol and peroxide;
110c: a crosslinked nanoparticle solution; and
110d: a mobile camera.
   Each of these items is described in detail in the following paragraphs. Please note that specific numerical values for concentrations, volumes, pH values, incubation times, and the like, are specified for purposes of illustration, and are not intended to be understood as limiting features.

The CL assay plate 110a includes, for example, a black microtiter plate containing multiplicity of wells, each having a sample volume of, say, 200 microliters (μL). The CL plate is bio-functionalized as follows. Each well is coated with a base linking material, such as gelatin in a carbonate buffer, e.g. 1% v/v gelatin in a 50 millimoles/liter (mmol/L) carbonate buffer having a pH of 9.6, and incubated for a period of two hours. The wells are then vigorously rinsed with a phosphate buffer saline (PBS) solution, having a concentration of 50 mmol/L and a pH of 7.4. After two consecutive washings with ultrapure water, the wells are incubated, for example, with glutaric dialdehyde (2.5% wt) solution for a period of 30 minutes. After another washing with ultrapure water, a pre-determined volume, of say 100 microliters (μL), of Hb stock solution of 1 microgram per milliliter (μg/mL) is added to each well, followed by crosslinking in PBS solution for a period of one hour, and a post-cleaning process.

The CL solution 110b contains luminol (C₈H₇N₃O₂), having a typical concentration of 0.45 mmol/L, and a colloidal suspension of nanoparticles (NPs). The NPs increase the sensitivity of the CL assay by enhancing the emission of CL light. The NPs may be prepared by the Turkevich method, which is familiar to those skilled in the art of preparing colloidal gold suspensions. NP diameters of 38 nanometers (nm) or less have been found to provide the greatest enhancement of light emission. The luminol solution may be mixed with a sodium hydroxide (NaOH) solution (15 mmol/L) for pH control and with a material such as 1,3-propanedithiol (PDT) solution in methanol, to facilitate crosslinking of the NPs.

The peroxide solution 110c is hydrogen peroxide (H₂O₂) having a typical concentration of 0.001 mmol/L.

The camera 110d is typically a miniature camera which may be integrated into a smartphone, such as an i-phone made by Apple Inc. or one of the many android-based phones available from Samsung, Google, and other manufacturers. To reduce the amount of stray light, relative to the CL-generated light, entering the camera, the camera may be fitted with a shroud and/or with a selective blue filter which attenuates light having wavelengths that fall outside the typical CL wavelength range of approximately 400 to 450 nm.

The camera 110d may be implemented as an image sensor which includes, for example, a charge-coupled device (CCD) sensor, a complementary metal-oxide-semiconductor (CMOS) sensor, or a photomultiplier.

In addition to the kit items 110a-110d, there should also be available a source of purified water for preparing different milk sample dilutions, and a sample of healthy milk, having little or no Hp protein, that may be used as a control.

Returning to the system 100 of FIG. 1, step (a) consists of drawing milk from a bovine quarter which is under inspection and preparing milk sample dilutions 120 having a variety of dilution ratios by mixing with purified water. The dilutions are identified as 1 part milk to X parts water, where X may be, for example, 0 (no dilution), 1, 10, or 20 fold dilution.

Each well of assay plate 110 is filled with a specific volume, of say 200 μL, of sample solution. At least one well, labelled “H” in FIG. 1, contains a dilution of healthy milk as a control. The other wells contain various dilutions of the milk under test. The samples are left to react with the Hb-modified wells for a time interval 130, which may be for example 20 to 30 minutes.

In step (b) of FIG. 1, controlled volumes of the CL solution 110b and of the peroxide solution 110c are applied to each of the wells in the plate 110, causing the emission of CL blue light, in a wavelength range that includes 425 nm. After a time interval 140, of for example ten seconds up to ten minutes, the CL light intensity has reached a steady-state, and the assay plate 110a is ready for imaging.

In step (c), the camera 110d is placed at a fixed height above the plate 110a, so that all the wells in the plate are contained within the field-of-view of the camera. Small deviations in the positioning of the camera from one image to the next may be compensated in image processing by identifying regions of interest, as will be explained below in regard to box (d). The intensity of the image pixels corresponding to a given well in plate 110a is proportional to the number of CL photons 150 emitted by each well per second, multiplied by the image exposure time in seconds.

After acquiring and storing one or more CL images 160, the images are transmitted to a digital signal processor 170 for image processing. The processor hardware and software may be contained and/or executed in the smartphone containing the camera 110d, or alternatively, in a remote computer or in a “cloud” computing environment.

The image processing in box (d) of FIG. 1 includes several algorithmic steps, as follows:
(i) Identification of regions of interest (ROIs) within the CL image 160; these are indicated by the dashed circles 165 overlaid on image 160 and correspond to the locations of wells on plate 110a;
(ii) Measuring the average CL intensity for the pixels within each of the ROIs;
(iii) Generating and storing in the processor 170 a regression curve 180 relating CL intensity to Hp level 175 (in units of μg/mL), for use in estimating the Hp level in any given milk sample; and
(iv) Providing a BM clinical diagnosis of a given milk sample based upon the Hp concentration measurement.
   The regression curve in (iii) may be linear, as shown in FIG. 1, or more generally, it may be a non-linear curve. The BM clinical diagnosis in (iv) may be for example a determination of whether the Hp concentration in the milk sample indicates no significant BM disease or a level of sub-clinical or clinical BM disease.

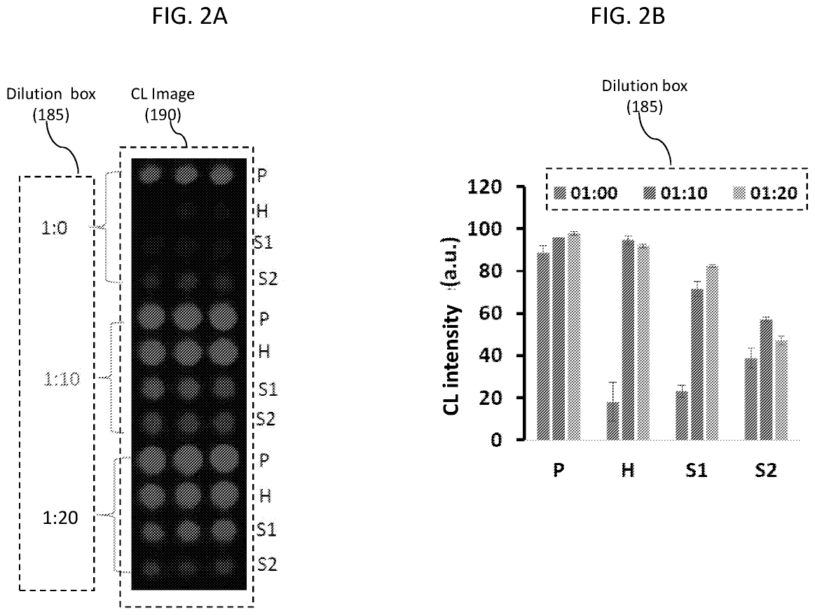

FIG. 2A shows an exemplary CL image 190 of an assay plate in which each row has three wells containing the same sample type, and different rows have different sample types. Sample type P represents pure water and sample type H denotes healthy milk having little or no Hp protein. Samples S1 and S2 have differing somatic cell counts (SCCs) per mL, as shown in Table 1 below.

TABLE 1

| | SCC (×10³) | |
|---|---|---|
| Milk sample | cells/mL | Bacteria |
| P | 0 | N/A |
| H | <90 | N/A |
| S1 | 300 | Streptococcus |
| | | dysgalactiae |
| S2 | 600 | Streptococcus |
| | | dysgalactiae |

The samples in rows 1-4, at the top of the CL image 190, are undiluted, as denoted by “1:0” in the dilution box 185. Similarly, rows 5-8 and rows 9-12 correspond to dilution ratios of 1:10 and 1:20, respectively.

Within each set of 4 rows corresponding to a given dilution, sample P has the highest CL emission intensity, because it has zero Hp protein and therefore no inhibition of the CL emission due to Hp-Hb binding. Samples H, S1, and S2 have increasing amounts of Hp protein, and therefore successively decrease the CL emission intensities, due to increased Hp-Hb binding.

FIG. 2B shows an exemplary graph of measured CL intensities, in arbitrary units (a.u.), versus sample type (P, H, S1, and S2) and dilution level (1:0, 1:10, and 1:20). For dilution 1:0 (e.g. no dilution, in blue), there is essentially no ability to discern intensity differences between samples H, S1, and S2. For dilutions 1:10 (in red) and 1:20 (in green), there is a significant intensity difference between samples H, S1 and S2. In fact, for the 1:10 dilution level, the CL intensity goes from 95 for H to 70 for S1 and to 55 for S2. This enables very good discrimination between the corresponding SCC levels, of 90, 300 and 600 cells per mL, shown in Table 1.

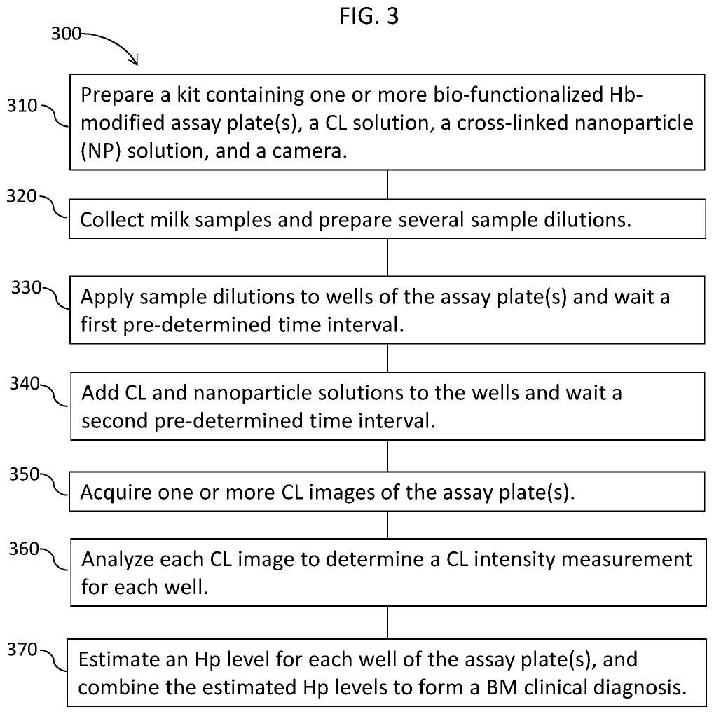

FIG. 3 shows an exemplary block diagram of the method for early detection of BM using enhanced chemiluminescence, according to the invention. The method consists of the following sequential steps:
Step 310: Preparation of a kit containing one or more bio-functionalized Hb-modified assay plate(s) each with a multiplicity of wells, a CL solution, a cross-linked nanoparticle solution, and a camera;
Step 320: Collection of milk samples on-site and preparation of several sample dilutions, for example, with purified water;
Step 330: Application of sample dilutions to the wells of an assay plate(s), and waiting a first pre-determined time interval for an Hp-Hb binding reaction to occur;
Step 340: Addition of the CL and nanoparticle solutions to the wells, and waiting a second pre-determined time interval for a CL emission intensity to approach a steady-state;
Step 350: Acquisition of one or more CL images of the assay plate(s), using the camera;
Step 360: Analysis of each CL image to determine a CL intensity measurement for each well; and
Step 370: Estimation of an Hp level for each well and combining the estimated Hp levels to form a BM clinical diagnosis.

The estimation in step 370 may utilize a pre-determined regression curve, such as curve 180 shown in box (d) of FIG. 1.

The descriptions of the various embodiments of the present disclosure have been presented for purposes of illustration, but are not intended to be exhaustive or limited to the embodiments disclosed. For example, the nanoparticles (NPs) may or may not be crosslinked with PDT. Furthermore, the NPs of the CL solution may be nanoparticles of various highly reflective materials, such as gold, magnetite (Fe₃O₄) or zinc oxide (ZnO), or other nanoscale materials with catalytic activity (e.g. ions, enzymes), all of which may enhance the emission of CL light produced in the CL assay.

Many other modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method for early detection of Bovine Mastitis using enhanced chemiluminescence, the method comprising the steps:
a) Preparing one or more bio-functionalized Hemoglobin (Hb)-modified assay plates, a chemiluminescent (CL) solution, a crosslinked nanoparticle solution and a camera;
b) Collecting milk samples and preparing a multiplicity of sample dilutions;
c) Applying sample dilutions to wells of the assay plate(s) and waiting a first pre-determined time interval;
d) Adding the CL and nanoparticle solutions to the wells and waiting a second pre-determined time interval;
e) Acquiring one or more CL images of the assay plate(s);
f) Analyzing each CL image to determine a CL intensity measurement for each well; and
g) Estimating a Haptoglobin (Hp) level for each well of the assay plate(s) and combining the estimated Hp levels to form a BM clinical diagnosis.

2. The method of claim 1 wherein the CL solution comprises luminol and/or peroxide solutions.

3. The method of claim 1 wherein the nanoparticle solution comprises one or more materials selected from a group consisting of gold, magnetite (Fe₃O₄) and zinc oxide (ZnO).

4. (canceled)

5. The method of claim 1 wherein the camera is sensitive to CL blue light emitted in a wavelength range that includes 425 nanometers.

6. The method of claim 1 wherein the camera is fitted with a selective blue filter and/or a shroud.

7. The method of claim 1 wherein the sample dilutions differ in dilution ratio by at least an order of magnitude.

8. The method of claim 1 wherein at least one of the wells of the assay plate(s) is a control well, corresponding to no significant BM disease.

9. The method of claim 1 wherein an Hp-Hb binding reaction occurs during the first pre-determined time interval.

10. (canceled)

11. The method of claim 1 wherein a CL emission intensity approaches a steady-state during the second pre-determined time interval.

12. (canceled)

13. The method of claim 1 wherein the CL intensity measurement is determined by averaging over a region of interest in the CL image.

14. The method of claim 1 wherein the estimating of the Hp level utilizes a pre-determined regression curve.

15. The method of claim 1 wherein the BM clinical diagnosis corresponds to a BM disease level selected from a group consisting of no significant disease, sub-clinical disease, and clinical disease.

16. A system for early detection of Bovine Mastitis using enhanced chemiluminescence, the system comprising:
one or more bio-functionalized Hemoglobin (Hb)-modified assay plate(s), each plate having a multiplicity of wells containing different milk sample dilutions;
a chemiluminescence (CL) solution and a crosslinked nanoparticle solution for producing an emission of CL light by the milk sample dilution in each of the wells;
a camera configured to receive the emission of CL light and to produce CL images; and
a processor;
wherein the processor is configured to analyze the CL images to determine a CL intensity measurement and an estimate of Haptoglobin (Hp) level, for each well of the assay plate(s).

17. The system of claim 16 wherein the CL solution comprises luminol and/or peroxide solutions.

18. The system of claim 16 wherein at least one of the wells of the assay plate(s) is a control well, corresponding to no significant BM disease.

19. (canceled)

20. The system of claim 16 wherein the processor utilizes a pre-determined regression curve to determine the estimates of Hp level.

21. The system of claim 16 wherein the processor is configured to combine the estimates of Hp level to form a BM clinical diagnosis.

22. The system of claim 21 wherein the BM clinical diagnosis corresponds to a BM disease level selected from a group consisting of no significant disease, sub-clinical disease, and clinical disease.

23. The system of claim 16 wherein the processor and the camera are integral components of a smartphone.

24. The system of claim 16 wherein the camera incorporates a charge-coupled device (CCD) sensor, a complementary metal-oxide-semiconductor (CMOS) sensor, or a photomultiplier.
